(19) United States
(12) Patent Application Publication
(10) Pub. No.: US 2025/0347668 A1
(43) Pub. Date: Nov. 13, 2025

(54) NOVEL TECHNIQUE TO QUANTIFY GASEOUS REACTIVE CHLORINE SPECIES BY LIQUID ION CHROMATOGRAPHY

(71) Applicant: THE REGENTS OF THE UNIVERSITY OF CALIFORNIA, Oakland, CA (US)

(72) Inventors: John Koster, Santa Cruz, CA (US); Soren Tornoe, Santa Cruz, CA (US); Donald Potts, Santa Cruz, CA (US); Nobuhiko Kobayashi, Santa Cruz, CA (US)

(73) Assignee: THE REGENTS OF THE UNIVERSITY OF CALIFORNIA, Oakland, CA (US)

(21) Appl. No.: 18/869,438

(22) PCT Filed: Jun. 1, 2023
(86) PCT No.: PCT/US2023/067751

§ 371 Date: Nov. 26, 2024

Related U.S. Application Data
(60) Provisional application No. 63/347,891, filed on Jun. 1, 2022.
(60) Provisional application No. 63/425,632, filed on Nov. 15, 2022.
(60) Provisional application No. 63/425,635, filed on Nov. 15, 2022.

(51) Int. Cl. G01N 30/96; G01N 30/02; G01N 30/62; G01N 30/88; G01N 33/00

(52) U.S. Cl. CPC G01N 30/96; G01N 30/62; G01N 30/88; G01N 33/0052; G01N 2030/027; G01N 2030/8809

(57) ABSTRACT

An apparatus useful for the collection (and optionally measurement of) of an amount of a first fluid, including a vial comprising an opening and a septum covering the opening. The septum is pierced by a needle configured for coupling to a source suspected of outputting the fluid. The vial further comprises a second fluid capable of combining with (e.g., dissolving) the first fluid (e.g., gas) introduced into the vial via the needle, e.g., to form a solution. The apparatus further includes an incubator capable of chilling the vial and its contents to between 10° C. and −5° C. when the vial is positioned within the incubator such that the septum faces the ground or a bottom of the incubator. A liquid ion chromatography instrument can then be used to quantify the amount of moiety of the substance of the first fluid.

### CROSS REFERENCE TO RELATED APPLICATIONS

This application claims the benefit under 35 U.S.C. Section 119(e) of the following co-pending and commonly-assigned applications:

U.S. Provisional Application Ser. No. 63/347,891, filed on Jun. 1, 2022, by John Koster, Soren Tornoe, Donald Potts, and Nobuhiko Kobayashi, entitled “GREEN HYDROGEN FROM SEAWATER,” Attorney's Docket Number 284.0012USP1; and

U.S. Provisional Application Ser. No. 63/425,632, filed on Nov. 15, 2022, by John Koster, Soren Tornoe, Donald Potts, and Nobuhiko Kobayashi, entitled “USING ARRAYS OF MICROELECTRODES IN ELECTROLYSIS TO TAILOR ELECTROCHEMICAL REACTIONS BY HIGHER ELECTRICAL CURRENT DENSITY AND ELECTRICAL POTENTIAL GRADIENT,” Attorney's Docket Number 284.0012USP2; and

U.S. Provisional Application Ser. No. 63/425,635, filed on Nov. 15, 2022, by John Koster, Soren Tornoe, Donald Potts, and Nobuhiko Kobayashi, entitled “NOVEL TECHNIQUE TO QUANTIFY GASEOUS REACTIVE CHLORINE SPECIES BYLIQUID ION CHROMATOGRAPHY,” Attorney's Docket Number 284.0016USP1;

all which applications are incorporated by reference herein.

This application is related to PCT international application serial No. XXX/XXXX/XXXXX, filed on same date herewith, by John Koster, Soren Tornoe, Donald Potts, and Nobuhiko Kobayashi, entitled “GREEN HYDROGEN FROM SEAWATER,” Attorney's Docket Number 284.0012WOU1, which application claims the benefit under 35 U.S.C. Section 119 (e) of the following co-pending and commonly-assigned applications:

U.S. Provisional Application Ser. No. 63/347,891, filed on Jun. 1, 2022, by John Koster, Soren Tornoe, Donald Potts, and Nobuhiko Kobayashi, entitled “GREEN HYDROGEN FROM SEAWATER,” Attorney's Docket Number 284.0012USP1; and

U.S. Provisional Application Ser. No. 63/425,632, filed on Nov. 15, 2022, by John Koster, Soren Tornoe, Donald Potts, and Nobuhiko Kobayashi, entitled “USING ARRAYS OF MICROELECTRODES IN ELECTROLYSIS TO TAILOR ELECTROCHEMICAL REACTIONS BY HIGHER ELECTRICAL CURRENT DENSITY AND ELECTRICAL POTENTIAL GRADIENT,” Attorney's Docket Number 284.0012USP2;

which applications are incorporated by reference herein.

### BACKGROUND OF THE INVENTION

### 1. Field of the Invention.

The present invention relates to methods and systems for the collection and measurement of halogens or other analytes/species/substances in fluid form.

### 2. Description of Related Art

Standard techniques for analysis of halogens include titrations, calorimetry, and mass spectrometry which are either cumbersome or lacking in precision for some desired applications (e.g. detection of chlorine evolution in electrolysis of seawater). What is needed are improved methods for the collection and measurement of halogens. The present invention satisfies this need.

### SUMMARY OF THE INVENTION

A highly sensitive apparatus for precise quantification of substances (e.g., halogens or halides, e.g., chlorinous gases).

Illustrative examples of the inventive subject matter disclosed herein include, but are not limited to, the following examples.

1. A device, comprising:

a vial comprising an opening, said opening covered by a septum (or a septum for covering the opening), a needle configured to be coupled to a source suspected of outputting a first fluid; and wherein:

said vial comprises or is primed to receive a second fluid capable of combining with the first fluid introduced into the vial when the needle is coupled to the source and the septum covering the opening is pierced by the needle (or the needle is inserted through or passes through the septum); and

an incubator capable of chilling the vial and the second fluid therein to between 10° C. and −5° C. when the vial is positioned within the incubator such that the septum faces the ground or a bottom of the incubator.

2. The device of example 1, wherein the first fluid is a halogenous fluid.

3. The device of example 2, wherein the halogenous fluid is selected from Cl₂, Br₂, and I₂ or comprises a halide comprising a chloride, a bromide, or an iodide.

4. The device of example 2, wherein the halogenous fluid comprises at least one of a halogenous gas or halogenous ion.

5. The device of any of example 4, wherein the second fluid comprises a solvent or eluent consisting essentially of, or comprising, milli-Q® ultra-pure water, a hydroxide, a carbonate, or a methanesulfonic acid (MSA), or an eluent used in a chromatography system.

6. The device of example 4, wherein the second fluid comprises a solvent capable of at least dissolving or absorbing the halogenous gas or halogenous ion.

7. The device of example 6, wherein the incubator comprises a coolant.

8. A system, comprising:

the device of the example 4; and

an apparatus measuring an amount of the halogenous gas or halogenous ion in the second fluid when the second fluid is received in the apparatus.

9. The system of example 9, wherein the apparatus comprises a liquid ion chromatography instrument.

10. The system of example 9, wherein the vial is positioned below the source so as to form a pressure gradient promoting combination of the halogenous gas or halogeneous ion with the second fluid

11. A system comprising the device of example 1 and the source coupled to the needle via a conduit through which the first fluid is transported from the source to the needle.

12. The device of example 1, wherein the vial contains the second fluid at a pressure of greater than 1 atmosphere at sea level.

13. A method of measuring a gas, comprising:

coupling a container to a source suspected of producing a gas;

collecting the gas produced by the source in the container comprising a second fluid, wherein the gas combines with the second fluid; and

receiving the second fluid in a chromatography system; and

measuring, in the chromatography system, an amount of the gas combined with the second fluid.

14. The method of example 13, wherein the source is an apparatus for electrolysis of seawater and the gas comprises a halogenous gas.

15. The method of example 13, wherein the gas comprises at least one of a fluoride, a chloride, a nitrite, a sulfate, a bromide, a nitrate, a phosphate, an acetate, a formate, a chlorite, a carbonate, or a chlorate.

16. The method of example 13, wherein the gas collection apparatus comprises:

a vial comprising an opening, said opening covered by a septum;

a needle coupled to the source and piercing the septum;

the vial comprising the second fluid capable of combining with the gas introduced into the vial via the needle; and

an incubator chilling the vial and the second fluid therein to between 10° C. and −5° C.; and

the vial positioned within the incubator such that the septum faces the ground or a bottom of the incubator.

17. The method of example 16, further comprising, prior to collecting the gas:

flushing the vial with the second fluid to remove any residual traces of a composition of the gas in the container; and

filling the vial to the brim with the second fluid and attaching a septum covering an opening in the container so that the second fluid forces the septum to bulge, thereby ensuring no air trapped within the vial.

18. The method of example 16, wherein the chromatography system comprises an ion chromatography system comprising a column, the method further comprising:

prior to the collecting, flushing the column with the second fluid until an output of the instrument is linear and a peak, corresponding to a threshold amount of the gas is below a predetermined value; and

receiving the second fluid from the vial;

loading the second fluid from the container into the flushed column; and

measuring the amount of the gas using the ion chromatography.

19. The method of example 13, wherein the source comprises a refrigeration system, an air conditioning system, a system for cleaning metal or electronic components, a system performing an electrochemical reaction, or an electrolyzer performing electrolysis.

20. The method of example 13, wherein the second fluid consists essentially of, or comprises at least one of, Type 1 ultrapure water, a hydroxide, carbonate, methanesulfonic acid (MSA), or an eluent for the gas used in the chromatography instrument.

21. An apparatus useful for the collection of a gas, comprising:

means for collecting a gas from a source suspected of outputting the gas and combining the gas with a fluid;

means for outputting the fluid to a system capable of measuring the gas in the fluid.

22. The apparatus of example 20, wherein:

the means for collecting comprises a container containing the fluid; a septum; a conduit or line comprising a passage passing through the septum into the container, wherein the septum seals the vial except for the passage into the container through the conduit or the line.

23. The apparatus of example 20, wherein the system capable of measuring the gas comprises a liquid ion chromatography instrument configured to measure or detect at least one of an amount or composition of a particular species in the gas.

24. The apparatus of example 20, wherein the fluid is at a pressure of greater than 1 atmosphere at sea level.

In one example, the halogen comprises a gas evolved at the anode during high current density microscale seawater electrolysis. The gases are routed to an iced, sealed, Milli-Q water containing collection vial, where a proportion of the reactive chlorine species fall into solution. The resulting solute is then expeditiously injected into a liquid ion chromatography instrument, which provides a readout, measuring an amount of the chloride ion, in microsiemens.

### BRIEF DESCRIPTION OF THE DRAWINGS

Referring now to the drawings in which like reference numbers represent corresponding parts throughout:

FIG. 1A. Experimental setup for collection of gases evolved from electrolysis of seawater using continuous application of current density larger than 1 Amp per centimeter square.

FIG. 1B. Example screw cap septum vial for collecting the halogen.

FIG. 2. Flowchart illustrating a method of configuring and assembling an apparatus used for collecting a halogen moiety for subsequent measurement.

FIG. 3. Flowchart illustrating a method of collecting and measuring a halogen.

FIG. 4A. Flowchart illustrating a method of collecting and measure a gas, and a method of making the system for collecting and measuring the gas.

FIG. 4B: Chlorine concentration evolved using the apparatus of FIG. 1A to perform electrolysis of seawater, showing the dependence of the relative chlorine concentration [Cl] on Jc.

FIG. 5. Example system comprising the source, collector, chromatography system, and computer.

FIG. 6. Example Hardware environment for controlling, measuring, or monitoring the fluids/gases outputted from the source.

FIG. 7. Example Network environment for controlling. Measuring, or monitoring the fluids/gases outputted from the source.

### DETAILED DESCRIPTION OF THE INVENTION

In the following description of the preferred embodiment, reference is made to the accompanying drawings which form a part hereof, and in which is shown by way of illustration a specific embodiment in which the invention may be practiced. It is to be understood that other embodiments may be utilized and structural changes may be made without departing from the scope of the present invention.

### Technical Description

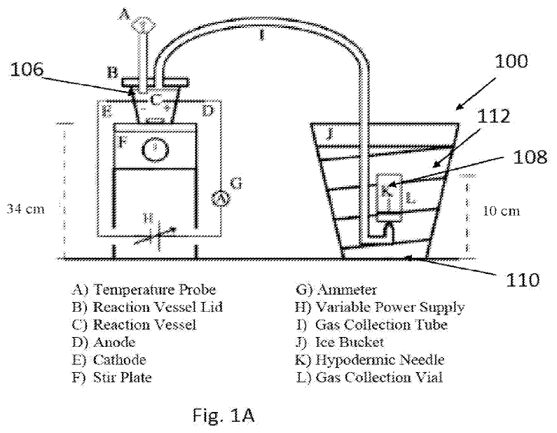

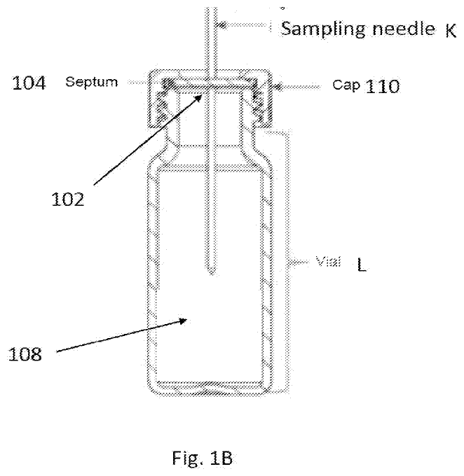

The present disclosure describes an apparatus useful for collecting and quantifying amounts of a chemical species/analyte such as a halogen. FIG. 1A illustrates an example apparatus comprising a vial L comprising an opening covered by a septum or cap, and a needle K piercing the septum or cap, wherein the needle is configured for coupling to a source C suspected of producing the halogen gas. The vial further comprises a solvent capable of dissolving the halogen gas introduced into the vial via the needle; and an incubator J capable of chilling the vial and the solvent therein to between 10° C. and −5° C. The vial is positioned within the incubator such that the septum faces the ground or the bottom of the incubator.

FIG. 1B illustrates an example vial comprising a glass screw cap septum vial L, comprising a cap 110 for securing the septum 104 covering the opening 102 in the vial, and a needle K piercing the septum. The septum may comprise a self-sealing PTFE/Silicon disc.

### Electrolysis Embodiment

a. Apparatus

The apparatus can be embodied in many ways. FIG. 1A illustrates a specific example used during electrolysis of seawater, wherein the screw-lid to the reaction vessel (A), used for performing electrolysis, was holed in its center, and a length of 5/16″ I.D. Nalgene 180 PVC tubing (VI Grade) cemented in place with E6000 adhesive (I). This tubing carries the combined evolved anodic and cathodic gases to the collection vial located at its distal end (L). The end of the tube is fitted with a fine insulin-type hypodermic needle (K), arranged so as to point upwards, which pierces the self-sealing septum of the collection vial used for collecting the evolved gases. The active ends of the electrodes (E, D) used for electrolysis were trimmed flush with a razor and polished with very fine sandpaper. The electrodes were then inserted through the holes on opposite sides of the reaction chamber.

In the illustrated example, the evolved gases collection vial is a 24 ml glass screw cap septum vial with self-sealing PTFE/Silicon disc, filled with Milli-Q water and suspended upside down in an ice bath (J). The Milli-Q water is very receptive to absorbing gases due to it being Type 1 ultrapure water. Similarly, the ice reduces the energy of the evolved gases, which helps them fall into solution. To further aid in the collection of the evolved gases, the reaction vessel is elevated roughly 20 cm above the evolved gases collection vial in order to push the pressure gradient in favor of gas absorption by the Milli-Q water.

b. Apparatus Configuration and Assembly

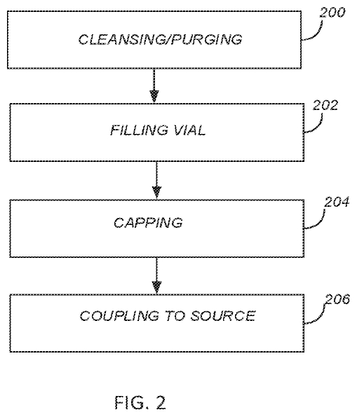

FIG. 2 is a flowchart illustrating a method of preparing the apparatus for a measurement. The method is illustrated in the context of preparing the apparatus for collecting gases evolved during electrolysis, but one of skill in the art understands the method can be adapted for collection of halogens/halides from other sources.

Block 200 represents cleansing/purging. The step comprises purging the liquid ion chromatography instrument (e.g., Dionex). In the electrolysis example, the Dionex was initially purged with Milli-Q water to flush its column, until the output was linear and the chlorine peak was below 0.02 microsiemens. Each run of the Dionex takes around 9 minutes to complete (at the machine's 35 mM Eluent Generator Target Concentration setting). In the electrolysis application, the step further comprised cleaning the reaction vessel (B), specifically, between runs or collections, scrubbing and rinsing the reaction vessel (B) to eliminate any residue. The step then further comprised partially filling the reaction vessel and/or priming the vial with liquid (e.g., .in tandem with the partial filling of the reaction vessel) to remove any residual traces of the chemical (halogen/halide) species being measured.

Block 202 represents completely filling the vial (to the brim). In the electrolysis example, the reaction vessel was filled with 120 ml of seawater electrolyte, leaving around 2 cm of head room between the electrolyte and the underside of the lid. In tandem, the collection vial was then primed (e.g., 3 times) with Milli-Q water to remove any residual traces of chemicals present in the vial. The vial was then filled to the brim with the solvent Milli-Q water).

Block 204 represents placing the septum over the vial and tightening the cap (securing the septum) down until the water forces the septum to bulge slightly, so as to ensure there is no air trapped within the vial.

Block 206 represents connecting the vial to a suspected halogen/halide source. In the example collecting halogen evolved during electrolysis, the evolved gases collection vial was inverted, its septum pierced with the insulin-needle at the end of the tubing for carrying the evolved gases, and then placed (upside down) in a bucket of ice.

c. Measurement Procedure

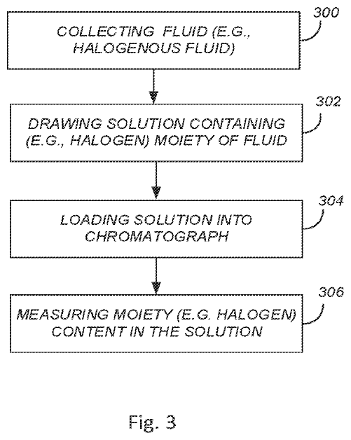

FIG. 3 is a flowchart illustrating a method of measuring an amount of the halogen, or halide, or halogenous fluid comprising or consisting essentially of the halide or halogen. The method is illustrated in the context of collecting gases evolved during electrolysis, but one of skill in the art understands the method can be adapted for collection of halogens or halides from other sources.

Block 300 represents switching on the halogen/halide source and allowing the apparatus to collect the halogen/halide in the liquid in the vial. For the example measurement of halogen gas evolved during electrolysis of seawater, the electrolysis was initiated by setting a desired voltage to the electrodes using a variable power supply (H) (the terminals of the power supply were first connected to the electrodes and then the power supply was switched from the stand-by setting to the “on” setting). The voltage was ramped over the duration of the electrolysis run (e.g., lasting 20 minutes, e.g., in 5.0 V increments from 10.0 to 90.0 Volts Direct Current).

In one or more examples, to maximize absorption in MilliQ water, the gas collection vial requires both pressurization and chilling (e.g., 760-1520 torr (i.e, 1-2 atm) at a temperature between 10° C. and −5° C.

Block 302 represents drawing a sample of the liquid, e.g., using a syringe. For the electrolysis example, at the end of each (e.g., 20-minute) electrolysis run, the evolved gases collection vial was detached, kept upside down to ensure no gas could escape, and a stand-alone syringe (e.g., hypodermic or insulin syringe) was primed with the resulting evolved gases solution to immediately draw a sample of the collected evolved-gases-in-Milli-Q water.

In some examples, in order to bring the concentration of gas samples from a particular process to within the range of the liquid IC's sensing capabilities, they may require to be either reduced or strengthened. This is learned through experimentation with the amount of time that the gas samples are fed into the collection vial, which can then be used as a procedural standard. The strengthening reducing procedures (if needed) may be applied using techniques known to those skilled in the art ordinary skill in the art (e.g., if the sample is outside the detection range of the system, dilution or concentration of the sample might be needed).

Block 304 represents loading the sample into a liquid ion chromatography system or apparatus for measuring the amount of the halogen/halide in the sample. In the electrolysis example, the step comprised loading the solution into the Dionex ICS-2000 Ion Chromatography System, fitted with a Dionex IonPac AS-18 IC column, capable of reporting the results (amount of halogen/halide) in micro-siemens.

Block 306 represents measuring the amount of the halogen/halide. For the electrolysis runs, the chloride ion peak occurred at the 3.97-minute mark on the resulting conductance graph outputted from the liquid ion chromatography instrument. Example halogens/halides include, but are not limited to, chlorine, bromine, iodine, chloride ions, bromide ions, iodide ions, or any other species, moiety, compound, or molecule comprising a halogen.

### Example: Collection and Measurement of Chloride Ions Produced in Electrolysis

### 1. Method

For each electrolysis run, the reaction vessel C of FIG. 1A was filled with 120 ml of electrolyte, leaving ˜2 cm of head room between the surface of the liquid and the underside of the screw lid. The electrolyte was coarsely filtered, natural seawater. The reaction vessel was placed on a magnetic stirring plate (“F”) and the electrolyte was stirred by a Teflon-covered magnet at ˜180 rpm throughout each run. The gas collection vial was filled to the brim with Milli-Q water and sealed by the septum in a way that ensured no air was trapped within the vial. The experiment were performed inside a fume hood maintained at room temperature and atmospheric pressure. Electrolysis was powered by a programmable DC power supply (Xantrex XFR 100-28, XFR 2800-Watt Series) (“H”) connected to the electrodes via an in-line ampere meter (Fluke 289 True-RMS multimeter) (“G”) for monitoring the electrical current. Each electrolysis run lasted 20 min, and was initiated by turning on the power, with the voltage preset to a desired value. At the 20 min mark, the power was shut off, and a sample of Milli-Q water containing the evolved mixed gases was immediately drawn with a 1.0 cc hypodermic syringe and loaded into a Dionex ICS-2000 liquid ion chromatography (IC) system with a Dionex IonPac AS-18 IC column. A series of electrolysis runs was carried out with specified voltages between 10 and 90 V with the corresponding Jc varying from ˜1 to ˜23 A cm⁻².

After each run, the reaction vessel was cleaned to remove any residue adhering to its inner walls, and the faces of the paired electrodes were refreshed (i.e., polished smooth). The gas collection vial was also flushed three times with Milli-Q water to remove any residual traces of chemicals present in the vial.

The IC system measured the electrical conductance of chloride (Cl) ions in microsiemens (μS) and used to measure chlorine concentration [Cl]. Before each analysis the IC system was given several Milli-Q water purges to flush its analysis column until the baseline chlorine ionic concentration stabilized below 0.02 μS. The concentration of eluent—the carrier fluid into which each sample was injected—was set to 35 mM, in reference to the concentrations of H⁺ and OH⁻ electrically generated by the liquid IC system.

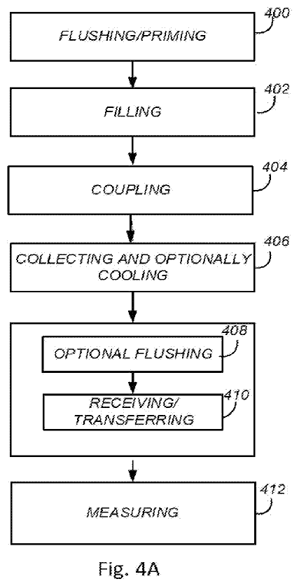

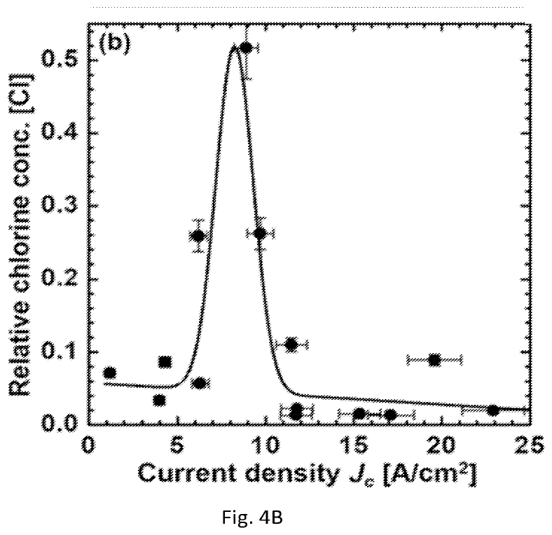

FIG. 4B shows Chlorine concentration evolved using the apparatus of FIG. 1A to perform electrolysis of seawater, showing the dependence of the relative chlorine concentration [C] on Jc.

### Example Systems

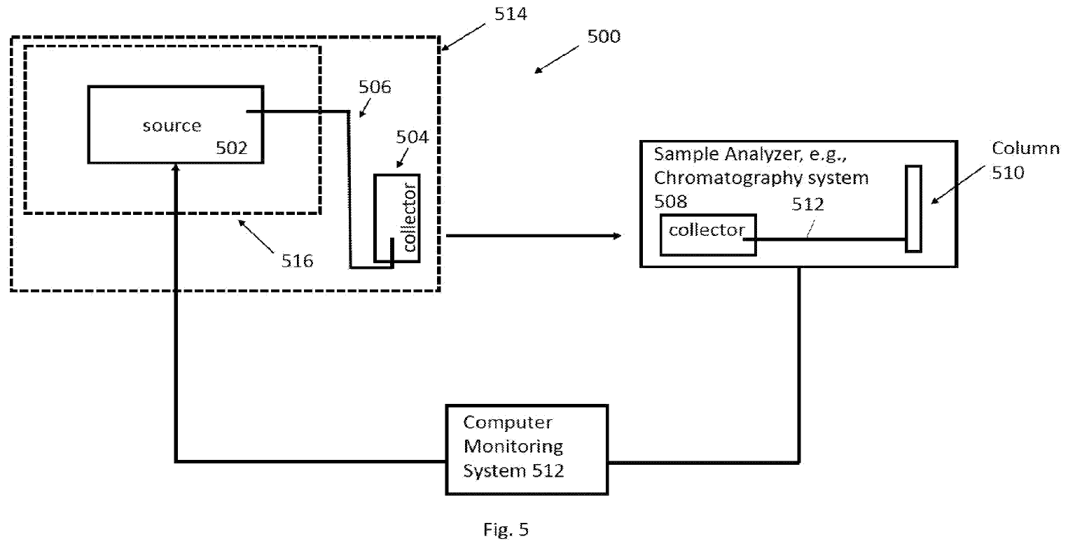

FIG. 5 illustrates an example system 500 comprising the source 502 (of the first fluid, e.g., gas) coupled to the collector 504 (e.g., vial L) via a conduit/conduit system 506 (e.g., comprising a tube I and needle K) through which the first fluid may flow or be transported to the collector. The collector, containing a second fluid for collecting the first fluid (e.g., gas) may be disconnected from the source and subsequently connected to a chromatography system 508 configured to measure the first fluid combined with the second fluid. A computer system monitoring an amount of the first fluid may provide feedback to the source, e.g., adjusting operation of, or deactivating, the source to control output of the first fluid depending on the amount first fluid measured in the chromatography system. For example, the source may be deactivated or otherwise adjusted to reduce output of the first fluid below a desired threshold level.

In one or more examples, the source 502 may comprise a refrigeration system, an air conditioning system, a system for cleaning metal or electronic components, a system performing an electrochemical reaction, or an electrolyzer performing electrolysis. The system 502 outputting various process gases or process fluids during operation may be coupled to the collector 504 for collecting process fluids/gases for subsequent measurement or monitoring in accordance with the methods described herein. In one or more examples, the collector 504 is connected to the source so that the source system 502 comprises the collector 504 (e.g., in the same space 514, e.g., room or ventilation space) containing the source. In yet further embodiments, the collector is connected to the source in a space 516 or region exterior to the source (e.g., a separate room, non-shared space).

### Possible Modifications and Variations

Although illustrated with halogenous fluids, the method and systems for quantifying many sorts of low concentration gases (i.e., not only those containing halogens), depending upon which of the various-purpose ion chromatography columns the liquid IC instrument has been fitted.

From the inventors' experience using an AS18 column, we can say with certainty that we can detect all the chemical/ions observed during our runs, which include (but are not limited to) Fluoride, Chloride, Nitrite, Sulfate, Bromide, Nitrate and Phosphate (Dionex literature regarding the AS18 also mentions Acetate, Formate, Chlorite, Carbonate and Chlorate, therefore these could be detected as well). The inventors expect the system to detect other chemicals/ions, and using an appropriate/correct column & paired eluent, one could detect just about anything in gaseous form that can be propelled into a gas collection vial.

Example columns (˜e.g., around 50) that can be used with the present invention include “Dionex IonPac polymeric columns” listed for other specific target applications [2]. The ThermoFisher Scientific catalog also mentions 3 different eluents (the solvent that removes the substance—MilliQ water is what is used with the AS18 column) [3]

Although MilliQ water is a good solvent for many gases, different type of solvent in the gas collection vial may be needed for certain other chemicals. Other categories of eluents include, but are not limited to, hydroxide, carbonate, and methanesulfonic acid (MSA) and types/derivatives thereof, and that may be used with the AS18 column [4].

### Device, System, and Method Embodiments

Illustrative methods, systems, and devices according to embodiments described herein include, but are not limited to, the following examples (referring also to FIGS. 1-7).

1. A device or apparatus 100 or kit, comprising:

a vial L comprising an opening 102, a septum for covering the opening, wherein said opening is optionally covered by a septum 104, and said septum is configured to be pierced by a needle K, wherein:

said needle is configured to be coupled to a source C suspected of outputting a first fluid 106; and

said vial further configured (e.g., primed) for containing a second fluid 108 capable of combining with the first fluid introduced into the vial via the needle (when the needle pierces, is inserted in, or passes through the septum); and

an incubator J capable of chilling the vial and the second fluid therein to between 10° C. and −5° C. when the vial is positioned within the incubator such that the septum faces the ground or a bottom 110 of the incubator.

2. The device of 1 where the needle is coupled to the source C via a tube I.

3. The device of examples 1 or 2, wherein the first fluid 106 is a halogenous fluid.

4. The device of example 3, wherein the halogenous fluid is selected from Cl₂, Br₂, and I₂ or comprises a halide comprising a chloride, a bromide, or an iodide.

5. The device of any of the examples 3-4, wherein the halogenous fluid comprises a halogenous gas.

6. The device of any of the examples 3-5, wherein the halogenous fluid comprises a halogenous ion.

7. The device of any of the examples 1-5 wherein the second fluid 108 comprises a solvent or eluent consisting essentially of, or comprising, milli-Q® ultra-pure water, .a hydroxide, a carbonate, or a methanesulfonic acid (MSA) or an eluent used in a chromatography system (e.g., liquid ion chromatography system).

8. The device of any of the examples 1-7, wherein the incubator is capable of chilling the vial and the second fluid to between 0° C. and 2° C.

9. The device of any of the examples 1-8, wherein:

the second fluid 108 comprises a solvent capable of dissolving the first fluid (e.g., halogenous fluid) comprising a gas to form a solution, or

the second fluid is capable of absorbing the first fluid (e.g., halogenous fluid) comprising a gas.

10. The device of any of the examples 1-9, wherein the incubator J comprises an ice-water mixture 112 or other refrigerant or coolant.

11. The device of any of the examples 1-10, wherein the septum is water-and gas-tight or fluid tight.

12. A system 500 or kit for measuring or detecting an amount of the first fluid (e.g., halogenous fluid), comprising:

the device 100 of any of the examples 1-11; and

an apparatus measuring an amount of the halogenous gas or halogenous ion in the second fluid when the second fluid is received in the apparatus; and/or

means for receiving 512 (e.g., insert, needle, conduit, tube) and analyzing (e.g., analyzer 508) the second fluid combined with the first fluid (e.g., halogenous fluid) and measuring the amount of the first fluid (e.g., halogenous fluid).

13. The system of example 11, wherein the means 508 comprises a liquid ion chromatography instrument configured to measure the amount of the first fluid (e.g., halogenous fluid).

14. The system of examples 12 or 13, wherein the vial L is positioned below the source C in order to form a pressure gradient promoting combination of the first fluid (e.g., halogenous fluid) with the second fluid.

15. A method of quantifying, measuring, or detecting the amount of a first fluid (e.g., halogenous fluid) produced by the source, the method comprising:

coupling a collection apparatus to the source suspected of producing the first fluid (e.g., halogenous fluid), the collection apparatus comprising a container (e.g., vial) containing a second fluid;

collecting the first fluid (e.g., halogenous fluid) produced by the source in the container (e.g., vial) comprising the second fluid, wherein the first fluid (e.g., halogenous fluid) combines with the second fluid, e.g., to form a third fluid; and

receiving the second fluid combined with the first fluid in an analyzer, and optionally analyzing 404 the third fluid/second fluid, so as to quantify an amount of the first fluid (e.g., halogenous fluid).

16. The method of example 15, wherein the source is designed to perform electrolysis of seawater.

17. The method of example 15 or 16, further comprising, prior to collecting the first fluid (e.g., halogenous fluid):

priming the container (vial) by flushing the vial with the second fluid to remove any residual traces of the first fluid (e.g., halogenous fluid) in the vial; and

filling the container (vial) to the brim with the second fluid and attaching a lid, cap, cover, or septum covering the opening so that the second fluid forces the septum, lid, cap, or cover to bulge, thereby ensuring no air trapped within the container (e.g., vial).

18. The method of any of the examples 15-17, wherein the analyzing comprises ion chromatography.

19. The method of example 18, further comprising performing the ion chromatography using a liquid ion chromatography instrument comprising a column, comprising:

prior to the collecting, flushing the column with the second fluid until an output of the instrument is linear and a peak, corresponding to a desired moiety/composition in the first fluid (e.g., halogen of the halogenous fluid) is below a predetermined value (e.g., 0.02 micro-siemens); and

drawing the third fluid from the vial;

loading the second fluid/third fluid into the flushed column; and

measuring the amount of the first fluid (e.g., halogenous fluid) using the ion chromatography.

20. The method of example 19, wherein the drawing uses a syringe.

21. The method or the device of any of the examples 1-20, wherein the source comprises a refrigeration system, an air conditioning system, a system for cleaning metal or electronic components, a system performing an electrochemical reaction, or an electrolyzer performing electrolysis.

22. The method of any of the examples 15-21, wherein the apparatus comprises the device of any of the examples 1-12.

23. The method or device of any of the examples 15-22, wherein the halogenous fluid comprises or consists essentially of one or more chloride ions.

24. The method or the device of any of the examples 1-23, wherein the second fluid consists essentially of, or comprises at least one of, Type 1 ultrapure water, a hydroxide, carbonate, or methanesulfonic acid (MSA).

25. The method of any of the examples 15-24, wherein the second fluid comprises a solvent for the halogenous fluid (e.g., gas) and the third fluid comprises a solution comprising the solvent and the halogenous fluid dissolved in the solvent.

26. The method or the device of any of the examples 2-25, wherein:

the tube I comprises PVC tubing (VI Grade); and

the vial comprises a glass screw cap septum vial L comprising a cap 110 for securing the septum 104, and

the septum 104 comprises a self-sealing PTFE/Silicon disc covering the opening.

27. The method or the device of any of the examples 1-26, wherein the source comprises a refrigeration system, an air conditioning system, a system for cleaning metal or electronic components, a system performing an electrochemical reaction, or an electrolyzer performing electrolysis (e.g., electrolysis of seawater form hydrogen).

28. FIG. 4A illustrates a method of measuring a gas, comprising:

coupling (Block 404) a container to a source suspected of producing a gas;

collecting (Block 406) the gas produced by the source in the container comprising a second fluid, wherein the gas combines with the second fluid;

optionally cooling (Block 406) the second fluid during the collecting;

transferring (Block 410) the second fluid in a chromatography system; and

measuring (Block 412), in the chromatography system, an amount of the gas combined with the second fluid.

29. The method of example 28, wherein the source is an apparatus for electrolysis of seawater and the gas comprises a halogenous gas.

30. The method of example 28, wherein the gas comprises at least one of a fluoride, a chloride, a nitrite, a sulfate, a bromide, a nitrate, a phosphate, an acetate, a formate, a chlorite, a carbonate, or a chlorate.

31. The method of any of the examples 28-30, wherein the gas collection apparatus comprises:

a vial comprising an opening, said opening covered by a septum;

a needle coupled to the source and piercing the septum;

the vial comprising the second fluid capable of combining with the gas introduced into the vial via the needle; and

an incubator chilling the vial and the second fluid therein to between 10° C. and −5° C.; and

the vial positioned within the incubator such that the septum faces the ground or a bottom of the incubator.

32. The method of any of the examples 28-30, further comprising, prior to collecting the gas:

Flushing/priming (Block 400) the vial with the second fluid to remove any residual traces of a composition of the gas in the container; and

filling the vial (Block 400) to the brim with the second fluid and attaching a septum covering an opening in the container so that the second fluid forces the septum to bulge, thereby ensuring no air trapped within the vial.

33. The method of any of the examples 28-32, wherein the chromatography system comprises an ion chromatography system comprising a column, the method further comprising:

prior to the collecting, and/or prior to the measuring, and/or prior to transferring, flushing (Block 408) the column with the second fluid until an output of the instrument is linear and a peak, corresponding to a threshold amount of the gas is below a predetermined value; and

receiving (Block 410) the second fluid from the vial;

loading (Block 410) the second fluid from the container into the flushed column; and

measuring (Block 412) the amount of the gas using the ion chromatography.

34. The method of any of the examples 28-33, wherein the source comprises a refrigeration system, an air conditioning system, a system for cleaning metal or electronic components, a system performing an electrochemical reaction, or an electrolyzer performing electrolysis.

35. The method of any of the examples 28-34, wherein the second fluid consists essentially of, or comprises at least one of, Type 1 ultrapure water, a hydroxide, carbonate, methanesulfonic acid (MSA), or an eluent for the gas used in the chromatography instrument.

36. An apparatus 500 useful for the collection of an amount of a first fluid (e.g., halogenous fluid), comprising:

means for collecting 504 a (e.g., halogenous) moiety and combining the (e.g., halogenous) moiety with a fluid;

means for outputting 512 (e.g., a needle, insert, conduit, tube) the fluid to a system 508 capable of measuring the (e.g., halogenous) moiety in the fluid.

37. An apparatus or kit or system or device useful for the collection of an amount of a first fluid (e.g., gas), comprising:

a container 504 (e.g., receptacle, flask, vial) configured for containing a fluid and/or primed for receiving the fluid; a lid, cap, cover, or septum; a conduit or line comprising a passage passing through the septum, lid, cap, or cover into the container, wherein the septum, lid, cap, or cover seals the container except for the passage into the container through the conduit or the line.

38. The apparatus of example 36 or 37, wherein the container or means for collecting comprises a vial L, the conduit comprises a needle K, and the means for outputting the fluid to the analyzing instrument comprises a needle, insert, or syringe or other dispenser for connecting to an autosampler or analyzing instrument (e.g., liquid ion chromatography instrument).

39. The apparatus of example 36, wherein the means for collecting comprises a container containing the fluid; a cover for the container; a conduit or line comprising a passage passing through the cover into the container, wherein the cover seals the vial except for the passage into the container through the conduit or the line.

40. The apparatus of any of the examples 2-38, wherein the system capable of measuring the (e.g., halogenous) fluid or gas comprises a liquid ion chromatography instrument, e.g., configured to measure or detect at least one of an amount or composition of a particular species in the gas.

41. The apparatus of any of the examples 1-40, further comprising a cooling system or incubator for cooling and/or incubating the fluid during the collecting by regulating temperature so that the gas combines with the fluid.

42. The apparatus, method, or system of any of the examples 1-41, wherein the ion chromatography instrument measures a concentration of a (e.g., halogenous) ionic species (e.g., chloride ions) by separating them based on their interaction with a resin in a column.

43. The apparatus of any of the example 36-42, wherein the means for collecting comprises the device of any of the examples 1-14.

44. The apparatus, method, or device of any of the examples 1-43, wherein the amount of the halogenous fluid comprises the amount, concentration, and/or composition of a particular (e.g., halogenous) moiety (e.g., halogen or halide) in the halogenous fluid.

45. The apparatus, method, or device of any of the examples 1-44, wherein the second fluid in the septum and/or vial is at a pressure of greater than 1 atmosphere at sea level.

46. The apparatus, method, or device of any of the examples 1-45, wherein the fluid comprises gas (e.g. halogenous gas) and water.

47. The apparatus, method, or device of any of the examples 1-46, wherein the vial or container comprises a chromatography vial [5-7] or medicine vial or container that can be disconnected from the vial or container and subsequently connected to the chromatography system so that the second fluid combined with the first fluid may be measured in the chromatography system.

48. The apparatus, method, or device of any of the examples 1-47, wherein the septum or cover comprises a resealable membrane that reseals after the needle, insert, or conduit is removed to maintain integrity of the contents of the vial or container and prevent contamination from external contaminants.

49. The apparatus, method, or device of any of the examples 1-48, wherein the vial or container comprises glass (e.g., borosilicate glass), plastic with a coating on the inner surfaces, the cap comprises plastic or metal, the cover or lid or septum covering the opening comprises rubber, elastomer, silicone, or PTFE, or metal.

50. The apparatus, method, or device of any of the examples 1-49, wherein the fluid or second fluid comprises the eluent or fluid used in the column of the liquid ion chromatography instrument.

51. A system comprising the device of any of the examples 1-14 and the source coupled to the needle via a conduit through which the first fluid is transported from the source to the needle.

52. The method of any of the examples 15-35 performed using the device, apparatus or system of any of the examples 1-14 or 36-51.

53. FIG. 4A illustrates a method of making a system for collecting and measuring a gas, comprising:

priming (Block 400) a container for receiving a second fluid capable of

combining with a sample fluid (first fluid or gas);

filling (Block 402) the container with the second fluid; and

coupling (Block 404) the container to a source suspected of producing a gas.

54. The method of example 53, wherein the system comprises the device of any of the examples 1-14 or 36-51.

55. The method or system of any of the examples 1-54, wherein the cap (e.g., screw cap) sealably secures the septum to cover the opening.

56. A device, comprising:

a vial L comprising an opening 102, said opening covered by a septum 104;

a needle K configured to be coupled to a source suspected of outputting a first fluid; and wherein:

said vial comprises or is primed to receive a second fluid capable of combining with the first fluid introduced into the vial when the needle is coupled to the source and the septum is pierced by the needle; and

an incubator J capable of chilling the vial and the second fluid therein to between 10° C. and −5° C. when the vial is positioned within the incubator such that the septum faces the ground or a bottom of the incubator.

57. The device or method of any of the examples 1-56 implemented with the device of example 56.

### Example Hardware Environment

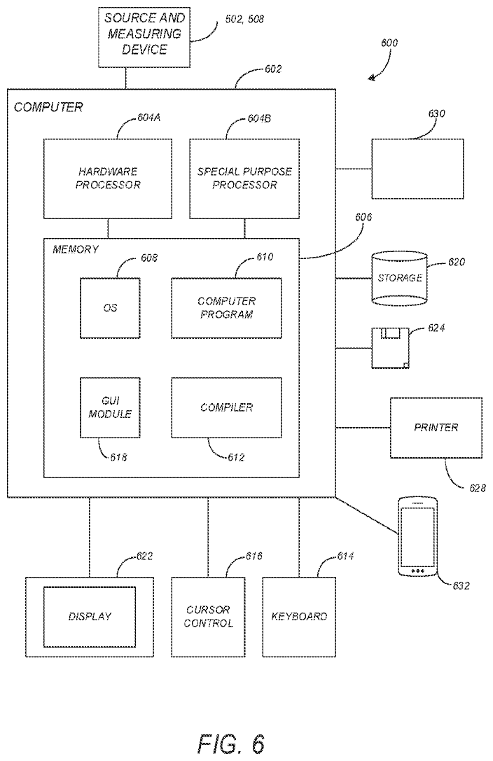

FIG. 6 is an exemplary hardware and software environment 600 (referred to as a computer-implemented system and/or computer-implemented method) used to implement one or more embodiments of the invention. The hardware and software environment includes a computer 602 and may include peripherals. Computer 602 may be a user/client computer, server computer, or may be a database computer. The computer 602 comprises a hardware processor 604A and/or a special purpose hardware processor 604B (hereinafter alternatively collectively referred to as processor 604) and a memory 606, such as random access memory (RAM). The computer 602 may be coupled to, and/or integrated with, other devices, including input/output (I/O) devices such as a keyboard 614, a cursor control device 616 (e.g., a mouse, a pointing device, pen and tablet, touch screen, multi-touch device, etc.) and a printer 628. In one or more embodiments, computer 602 may be coupled to, or may comprise, a portable device 632 (e.g., cellular device, mobile phone, laptop, tablet) or multi-touch device or other internet enabled device executing on various platforms and operating systems.

In one embodiment, the computer 602 operates by the hardware processor 604A performing instructions defined by the computer program 610 (e.g., an application) under control of an operating system 608. The computer program 610 and/or the operating system 608 may be stored in the memory 606 and may interface with the user and/or other devices to accept input and commands and, based on such input and commands and the instructions defined by the computer program 610 and operating system 608, to provide output and results.

Output/results may be presented on the display 622 or provided to another device for presentation or further processing or action. The image may be provided through a graphical user interface (GUI) module 618. Although the GUI module 618 is depicted as a separate module, the instructions performing the GUI functions can be resident or distributed in the operating system 608, the computer program 610, or implemented with special purpose memory and processors. In one or more embodiments, the display 622 is integrated with/into the computer 602 and comprises a multi-touch device having a touch sensing surface. Some or all of the operations performed by the computer 602 according to the computer program 610 instructions may be implemented in a special purpose processor 604B. In this embodiment, some or all of the computer program 610 instructions may be implemented via firmware instructions stored in a read only memory (ROM), a programmable read only memory (PROM) or flash memory within the special purpose processor 604B or in memory 606. The special purpose processor 604B may also be hardwired through circuit design to perform some or all of the operations to implement the present invention. Further, the special purpose processor 604B may be a hybrid processor, which includes dedicated circuitry for performing a subset of functions, and other circuits for performing more general functions such as responding to computer program 610 instructions. In one embodiment, the special purpose processor 604B is an application specific integrated circuit (ASIC) or field programmable gate array (FPGA).

The computer 602 may also implement a compiler 612 that allows an application or computer program 610 written in a programming language such as C, C++, Assembly, SQL, PYTHON, PROLOG, MATLAB, RUBY, RAILS, HASKELL, or other language to be translated into processor 604 readable code. Alternatively, the compiler 612 may be an interpreter that executes instructions/source code directly, translates source code into an intermediate representation that is executed, or that executes stored precompiled code. Such source code may be written in a variety of programming languages such as JAVA, JAVASCRIPT, PERL, BASIC, etc. After completion, the application or computer program 610 accesses and manipulates data accepted from I/O devices and stored in the memory 606 of the computer 602 using the relationships and logic that were generated using the compiler 612.

The computer 602 also optionally comprises an external communication device such as a modem, satellite link, Ethernet card, or other device for accepting input from, and providing output to, other computers 602.

In one embodiment, instructions implementing the operating system 608, the computer program 610, and the compiler 612 are tangibly embodied in a non-transitory computer-readable medium, e.g., data storage device 620, which could include one or more fixed or removable data storage devices, such as a zip drive, floppy disc drive 624, hard drive, CD-ROM drive, tape drive, etc. Further, the operating system 608 and the computer program 610 are comprised of computer program 610 instructions which, when accessed, read and executed by the computer 602, cause the computer 602 to perform the steps necessary to implement and/or use the present invention or to load the program of instructions into a memory 606, thus creating a special purpose data structure causing the computer 602 to operate as a specially programmed computer executing the method steps described herein. Computer program 610 and/or operating instructions may also be tangibly embodied in memory 606 and/or data communications devices 630, thereby making a computer program product or article of manufacture according to the invention. As such, the terms “article of manufacture,” “program storage device,” and “computer program product,” as used herein, are intended to encompass a computer program accessible from any computer readable device or media.

Of course, those skilled in the art will recognize that any combination of the above components, or any number of different components, peripherals, and other devices, may be used with the computer 602.

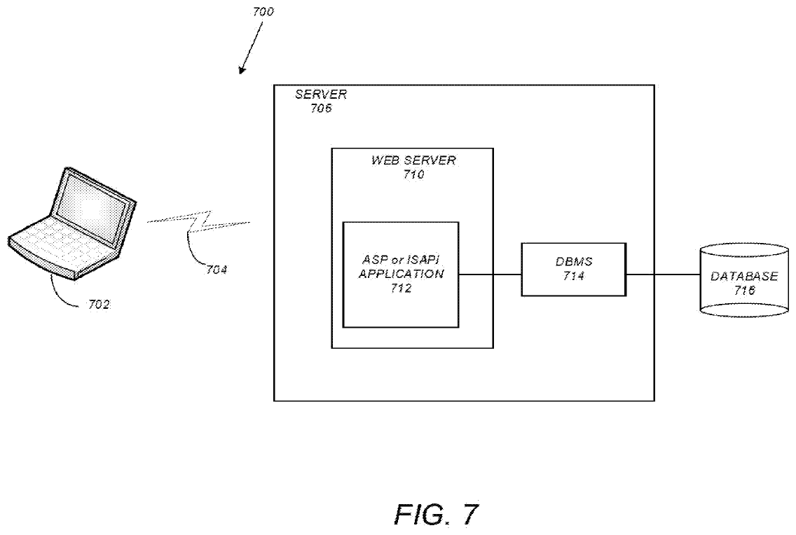

FIG. 7 schematically illustrates a typical distributed/cloud-based computer system 700 using a network 704 to connect client computers 702 to server computers 706. A typical combination of resources may include a network 704 comprising the Internet, LANs (local area networks), WANs (wide area networks), SNA (systems network architecture) networks, or the like, clients 702 that are personal computers or workstations (as set forth in FIG. 6), and servers 706 that are personal computers, workstations, minicomputers, or mainframes (as set forth in FIG. 6). However, it may be noted that different networks such as a cellular network or otherwise), or any other type of network may be used to connect clients 702 and servers 706 in accordance with embodiments of the invention.

A network 704 such as the Internet connects clients 702 to server computers 706. Network 704 may utilize ethernet, coaxial cable, wireless communications, radio frequency (RF), etc. to connect and provide the communication between clients 702 and servers 706. Further, in a cloud-based computing system, resources (e.g., storage, processors, applications, memory, infrastructure, etc.) in clients 702 and server computers 706 may be shared by clients 702, server computers 706, and users across one or more networks. Resources may be shared by multiple users and can be dynamically reallocated per demand. In this regard, cloud computing may be referred to as a model for enabling access to a shared pool of configurable computing resources.

Clients 702 may execute a client application or web browser and communicate with server computers 706 executing web servers 710. Such a web browser is typically a program such as MICROSOFT INTERNET EXPLORER/EDGE, MOZILLA FIREFOX, OPERA, APPLE SAFARI, GOOGLE CHROME, etc. Further, the software executing on clients 702 may be downloaded from server computer 706 to client computers 702 and installed as a plug-in or ACTIVEX control of a web browser. Web server 710 may host an Active Server Page (ASP) or Internet Server Application Programming Interface (ISAPI) application 712, which may be executing scripts.

Generally, these components 700-716 all comprise logic and/or data that is embodied in/or retrievable from device, medium, signal, or carrier, e.g., a data storage device, a data communications device, a remote computer or device coupled to the computer via a network or via another data communications device, etc. Moreover, this logic and/or data, when read, executed, and/or interpreted, results in the steps necessary to implement and/or use the present invention being performed.

Although the terms “user computer”, “client computer”, and/or “server computer” are referred to herein, it is understood that such computers 702 and 706 may be interchangeable and may further include thin client devices with limited or full processing capabilities, portable devices such as cell phones, notebook computers, pocket computers, multi-touch devices, and/or any other devices with suitable processing, communication, and input/output capability.

Of course, those skilled in the art will recognize that any combination of the above components, or any number of different components, peripherals, and other devices, may be used with computers 702 and 706. Embodiments of the invention are implemented as a software/application on a client 702 or server computer 706. Further, as described above, the client 702 or server computer 706 may comprise a thin client device or a portable device that has a multi-touch-based display.

### Advantages and Improvements

Quantification of Cl₂ gas evolved during saline water electrolysis is challenging because the Cl₂ is highly reactive. Standard techniques for analysis include titrations and calorimetry, which are either cumbersome or lacking in precision for the desired application. Moreover, for some applications where use of a specialized gas chromatograph or quadrupole mass spectrometer is not possible, a means of analysis via liquid ion chromatography may be desirable. The present invention satisfies these needs, providing a rapid process for quantifying halogen amounts with high sensitivity.

The apparatus described herein can be manufactured in a compact form (e.g., for coupling to a small ˜120 mL reaction chamber outputting the halogen gas, as used for electrolysis) from inexpensive components and robust off the shelf analytical instruments (e.g., a benchtop liquid ion chromatograph). Measurement readings can be obtained quickly (within 5 minutes of collection in the case of chloride). Alternatively, samples can be collected and stored for later analysis (e.g., by using funnels funneling the gases into a collection vial (full of Milli-Q water) with a sealable membrane).

### REFERENCES

The following reference is incorporated by reference herein.

[1] U.S. Pat. No. 5,279,167

[2] https://assets.thermofisher.com/TFS-Assets/CMD/brochures/st-70588-ic-column-selection-guide-st70588-en.pdf

[3] https://www.thermofisher.com/us/en/home/industrial/chromatography/ion-chromatography-ic/ion-chromatography-columns.html #:˜:text=Dionex%20IonPac%20columns%20are%20IonPac,systems%20using%20eluent%20generation%20cartridges

[4] https://www.thermofisher.com/us/en/home/industrial/chromatography/ion-chromatography-ic /ion-chromatography-columns/hydroxide-selective-anion-exchange-ic-columns.html.

[5] https://www.qecusa.com/chromatography.html?gclid=EAIaIQobChMIxuCr 1bmd_wIVswitBh014QNfEAAYAiAAEgKQRPD_BWE

[6] https://www.agilent.com/en/product/vials-sample-containment

[7] https://www.thermofisher.com/us/en/home/industrial/chromatography/chro matography-consumables/autosampler-vials-caps-hplc-gc.html?gclid=EAIaIQobChMIpvvd97md_wIVGgetBh2Uuw8mEAAYASAAEgIR3PD_BwE&cid=E.23CMD.DL102.12926.01&ef_id=EAIaIQobChMIpvvd97md_wIV GgetBh2Uuw8mEAAYASAAEgIR3PD_BWE: G: s&s_kwcid-AL!3652!3!596148161 476!e!!g!!thermo % 20vials&gad=

[8] https://www.thermofisher.com/us/en/home/industrial/chromatography/dion ex.html?gclid=EAIaIQobChMIlsiXudqg_wIV6RatBh33qQPCEAAYASAAEgK88fD BwE&cid=E.23CMD.DL103.12917.01&ef_id=EAIaIQobChMIlsiXudqg_wIV6Rat Bh33qQPCEAAYASAAEgK88fD_BwE: G: s&s kwcid=AL!3652!3!529706906839!p!!g!!dionex&gad=1

[9] Koster J W et al., Explicitly controlling electrical current density overpowers the kinetics of the chlorine evolution reaction and increases the hydrogen production during seawater electrolysis, International Journal of Hydrogen Energy, https://doi.org/10.1016/j.ijhydene.2022.11.053

### Conclusion

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

1. A device, comprising:
a vial comprising an opening, said opening covered by a septum;
a needle configured to be coupled to a source suspected of outputting a first fluid; and wherein:
said vial comprises or is primed to receive a second fluid capable of combining with the first fluid introduced into the vial when the needle is coupled to the source and the septum is pierced by the needle; and
an incubator capable of chilling the vial and the second fluid therein to between 10° C. and −5° C. when the vial is positioned within the incubator such that the septum faces the ground or a bottom of the incubator.

2. The device of claim 1, wherein the first fluid is a halogenous fluid.

3. The device of claim 2, wherein the halogenous fluid is selected from Cl₂, Br₂, and I₂ or comprises a halide comprising a chloride, a bromide, or an iodide.

4. The device of claim 2, wherein the halogenous fluid comprises at least one of a halogenous gas or halogenous ion. 5 (ORIGINAL) The device of any of claim 4, wherein the second fluid comprises a solvent or eluent consisting essentially of, or comprising, milli-Q® ultra-pure water, .a hydroxide, a carbonate, or a methanesulfonic acid (MSA), or an eluent used in a chromatography system.

6. The device of claim 4, wherein the second fluid comprises a solvent capable of at least dissolving or absorbing the halogenous gas or halogenous ion.

7. The device of claim 6, wherein the incubator comprises a coolant.

8. A system, comprising:
the device of the claim 4; and
an apparatus measuring an amount of the halogenous gas or halogenous ion in the second fluid when the second fluid is received in the apparatus.

9. The system of claim 9, wherein the apparatus comprises a liquid ion chromatography instrument.

10. The system of claim 9, wherein the vial is positioned below the source so as to form a pressure gradient promoting combination of the halogenous gas or halogeneous ion with the second fluid

11. A system comprising the device of claim 1 and the source coupled to the needle via a conduit through which the first fluid is transported from the source to the needle.

12. The device of claim 1, wherein the vial contains the second fluid at a pressure of greater than 1 atmosphere at sea level.

13. A method of measuring a gas, comprising:
coupling a container to a source suspected of producing a gas;
collecting the gas produced by the source in the container comprising a second fluid, wherein the gas combines with the second fluid;
cooling the second fluid during the collecting;
transferring the second fluid in a chromatography system; and
measuring, in the chromatography system, an amount of the gas combined with the second fluid.

14. The method of claim 13, wherein the source is an apparatus for electrolysis of seawater and the gas comprises a halogenous gas.

15. The method of claim 13, wherein the gas comprises at least one of a fluoride, a chloride, a nitrite, a sulfate, a bromide, a nitrate, a phosphate, an acetate, a formate, a chlorite, a carbonate, or a chlorate.

16. The method of claim 13, wherein the gas collection apparatus comprises:
a vial comprising an opening, said opening covered by a septum;
a needle coupled to the source and piercing the septum;
the vial comprising the second fluid capable of combining with the gas introduced into the vial via the needle; and
an incubator chilling the vial and the second fluid therein to between 10° C. and −5° C.; and
the vial positioned within the incubator such that the septum faces the ground or a bottom of the incubator.

17. The method of claim 16, further comprising, prior to collecting the gas:
flushing the vial with the second fluid to remove any residual traces of a composition of the gas in the container; and
filling the vial to the brim with the second fluid and attaching a septum covering an opening in the container so that the second fluid forces the septum to bulge, thereby ensuring no air trapped within the vial.

18. The method of claim 16, wherein the chromatography system comprises an ion chromatography system comprising a column, the method further comprising:
flushing the column with the second fluid until an output of the instrument is linear and a peak, corresponding to a threshold amount of the gas is below a predetermined value, and
receiving the second fluid from the vial;
loading the second fluid from the container into the flushed column; and
measuring the amount of the gas using the ion chromatography.

19. The method of claim 13, wherein the source comprises a refrigeration system, an air conditioning system, a system for cleaning metal or electronic components, a system performing an electrochemical reaction, or an electrolyzer performing electrolysis.

20. The method of claim 13, wherein the second fluid consists essentially of, or comprises at least one of, Type 1 ultrapure water, a hydroxide, carbonate, methanesulfonic acid (MSA), or an eluent for the gas used in the chromatography instrument.

21-25. (canceled)
